(19) United States
(12) Patent Application Publication
(10) Pub. No.: US 2024/0193729 A1
(43) Pub. Date: Jun. 13, 2024

(54) Systems and Methods for Synthetic Image Generation based on RNA Expression

(71) Applicant: The Board of Trustees of the Leland Stanford Junior University, Stanford, CA (US)

(72) Inventors: Francisco Carrillo-Perez, Stanford, CA (US); Marija Pizurica, Stanford, CA (US); Olivier Gevaert, Stanford, CA (US)

(73) Assignee: The Board of Trustees of the Leland Stanford Junior University, Stanford, CA (US)

(21) Appl. No.: 18/538,743

(22) Filed: Dec. 13, 2023

Related U.S. Application Data
(60) Provisional application No. 63/387,261, filed on Dec. 13, 2022.

(51) Int. Cl. G06T 3/4053; G06T 3/4046; G06T 5/50; G16B 35/20; G16B 40/00

(52) U.S. Cl. CPC G06T 3/4053; G06T 3/4046; G06T 5/50; G16B 35/20; G16B 40/00; G06T 2207/10056; G06T 2207/20016; G06T 2207/20081; G06T 2207/20084

(57) ABSTRACT

Systems and methods for synthetic image generation include a method of generating synthetic histological slide images that includes translating each of several RNA-Seq records into a latent space, training a first diffusion model to produce a first synthetic histological slide image at a lower resolution using the translated RNA-Seq records and associated histological slides, training a second diffusion model to upscale lower resolution synthetic histological slide images produced by the first diffusion model to higher resolution synthetic histological slide images, obtaining a given RNA-Seq record, translating the given RNA-Seq record into the latent space, providing the latent representation of the given RNA-Seq record to the trained first diffusion model to generate a given lower resolution synthetic histological slide image, and providing the given lower resolution synthetic histological slide image to the trained second diffusion model to generate a given higher resolution synthetic histological slide image.

### CROSS-REFERENCE TO RELATED APPLICATIONS

The current application claims the benefit of and priority under 35 U.S.C. § 119(e) to U.S. Provisional Patent Application No. 63/387,261 entitled “Systems and Methods for Synthetic Image Generation Based on RNA Expression” filed Dec. 13, 2022. The disclosure of U.S. Provisional Patent Application No. 63/387,261 is hereby incorporated by reference in its entirety for all purposes.

### STATEMENT OF FEDERALLY SPONSORED RESEARCH

This invention was made with Government support under contract CA260271 awarded by the National Institutes of Health. The Government has certain rights in the invention.

### FIELD OF THE INVENTION

The present invention generally relates to neuronavigation, and (more specifically) to generating personalized stimulation targets.

### BACKGROUND

Cancer is a disease in which certain cells in the body begin to grow uncontrollably and can spread to other parts of the body. These cells can form tumors and/or otherwise disrupt the body's natural processes which can lead to death. Pathologists use a variety of tools, including direct examination of tissue samples, to diagnose cancers. In pathology, whole-slide imaging (WSI, also known as virtual microscopy) refers to the scanning of glass slides are scanned to produce digital images. Tissue samples are typically placed on slides for imaging. Because the tissue samples are three-dimensional, WSI images may be multilayered to enable 3-dimensional digital representations. RNA sequencing (RNA-seq) is a process where input material (sometimes enriched for small RNAs) is sequenced for RNA fragments. RNA-seq enables examination of tissue-specific expression patterns, which can be helpful in cancer diagnostics.

In machine learning, variational autoencoders (VAEs) are artificial neural networks that function as probabilistic generative models. Typically, VAE structures include a probabilistic encoder and a probabilistic decoder as two neural network components. The first neural network maps the input to a latent space that corresponds to the parameters of a variational distribution. The decoder maps from the latent space to the input space.

### SUMMARY OF THE INVENTION

Systems and methods for synthetic image generation in accordance with embodiments of the invention are illustrated. One embodiment includes a method of generating synthetic histological slide images, including obtaining several RNA-Seq records, obtaining several histological slide images, where each histological slide image is associated with one of the RNA-Seq records, translating each record in the several RNA-Seq records into a latent space using an encoder component of a variational auto encoder, training a first diffusion model to produce a first synthetic histological slide image at a lower resolution using the translated several RNA-Seq records and the associated histological slides, training a second diffusion model to upscale lower resolution synthetic histological slide images to higher resolution synthetic histological slide images using lower resolution images produced by the first diffusion model and the associated histological slide images, obtaining a given RNA-Seq record, translating the given RNA-Seq record into the latent space using the encoder component of the variational autoencoder, providing the latent representation of the given RNA-Seq record to the trained first diffusion model, generating a given lower resolution synthetic histological slide image using the trained first diffusion model, providing the given lower resolution synthetic histological slide image to the trained second diffusion model, and generating a given higher resolution synthetic histological slide image using the trained second diffusion model.

In a further embodiment, the variational autoencoder is a B-VAE encoder model.

In still another embodiment, the method further includes steps for training the first and second diffusion models on a second plurality of RNA-Seq records, where each of the RNA-Seq records in the second plurality of RNA-Seq records are associated with one image of a second plurality of histological slide images, and where the second plurality of RNA-Seq records are associated with a specific cancer classification.

In a still further embodiment, the first and second diffusion models includes a UNet architecture.

In yet another embodiment, the lower resolution is 64×64 pixels.

In a yet further embodiment, the higher resolution is 256×256 pixels.

In another additional embodiment, the given higher resolution synthetic histological slide image is a tile of a larger synthetic histological slide image.

In a further additional embodiment, the method further includes steps for generating several higher resolution synthetic histological slide images, and combining the several higher resolution synthetic histological slide images to form the larger synthetic histological slide image.

In another embodiment again, the synthetic histological slide image depicts a plurality of human tissue types.

In a further embodiment again, the method further includes steps for training the encoder component of the variational autoencoder using a decoder component of the variational autoencoder, wherein the encoder component and the decoder component are trained together to minimize reconstruction error at the output of the decoder component.

One embodiment includes a system for generating synthetic histological slide images, including a processor, and a memory, the memory containing a whole-slide image synthesis application that configures the processor to obtain an RNA-Seq record, translate the RNA-Seq record into the latent space using an encoder component of a variational autoencoder, provide the latent representation of the RNA-Seq record to a first diffusion model, generate a lower resolution synthetic histological slide image using a trained first diffusion model, provide the given lower resolution synthetic histological slide image to a second diffusion model, and generate a given higher resolution synthetic histological slide image using the second diffusion model.

In still yet another embodiment, the encoder component is trained using a decoder component of the variational autoencoder, wherein the encoder component and the decoder component are trained together to minimize reconstruction error at the output of the decoder component.

In a still yet further embodiment, the first diffusion model and second diffusion model are trained by obtaining several RNA-Seq records, obtaining several histological slide images, where each histological slide image is associated with one of the RNA-Seq records, translating each record in the several RNA-Seq records into a latent space using an encoder component of a variational auto encoder, training a first diffusion model to produce a first synthetic histological slide image at a lower resolution using the translated several RNA-Seq records and the associated histological slides, and training a second diffusion model to upscale lower resolution synthetic histological slide images to higher resolution synthetic histological slide images using lower resolution images produced by the first diffusion model and the associated histological slide images.

In still another additional embodiment, the method further includes steps for training the first and second diffusion models on a second plurality of RNA-Seq records, where each of the RNA-Seq records in the second plurality of RNA-Seq records are associated with one image of a second plurality of histological slide images, and where the second plurality of RNA-Seq records are associated with a specific cancer classification.

In a still further additional embodiment, the variational autoencoder is a B-VAE encoder model.

In still another embodiment again, the first and second diffusion models includes a UNet architecture.

In a still further embodiment again, the lower resolution is 64×64 pixels.

In yet another additional embodiment, the higher resolution is 256×256 pixels.

In a yet further additional embodiment, the given higher resolution synthetic histological slide image is a tile of a larger synthetic histological slide image.

In yet another embodiment again, the method further includes steps for generating several higher resolution synthetic histological slide images, and combining the several higher resolution synthetic histological slide images to form the larger synthetic histological slide image.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is a flow chart of a training process for training a VAE encoder in accordance with an embodiment of the invention.

FIG. 2 is a flow chart of a training process for training an RNA-to-Image Diffusion Model and a Super-Resolution Diffusion Model in accordance with an embodiment of the invention.

FIG. 3 is a flow chart of a process for generating synthetic WSI images based on RNA-Seq data in accordance with an embodiment of the invention.

FIG. 4 graphically represents the process illustrated in FIG. 3 in accordance with an embodiment of the invention.

FIG. 5 illustrates real vs synthetic WSI images in accordance with an embodiment of the invention.

FIG. 6 is a block diagram for a WSI Synthesis device in accordance with an embodiment of the invention.

### DETAILED DESCRIPTION

Cancer is one of the leading causes of death worldwide. Recent advancements in medical technology have demonstrated the power of machine learning in the diagnostic process, particularly when provided with multi-modal data. For example, RNA-Seq, whole-slide imaging (WSI) and RNA-Seq, when provided together have significant diagnostic power. In particular, deep learning techniques have shown significant promise for cancer detection and classification when provided with this type of multi-modal data. A critical issue for these types of models is that they tend to need to be provided very large training data sets. Unfortunately, despite ongoing projects attempting to gather these data, sufficiently large training data sets are difficult to assemble. Often, clinicians have not performed every modality on a given sample, or failed to save it. While incomplete records have some value, they are inferior to a complete record as part of a training data set.

Systems and methods described herein can take any given RNA-Seq data and generate a synthetic WSI that approximates the tissue sample that produced said given RNA-Seq data with sufficient accuracy to train a machine learning model. In many embodiments, the encoder component of a variational autoencoder (VAE) is used to transform RNA-seq data into a latent space. In various embodiments, β-VAE is selected as the VAE, although other VAE models can be used as appropriate to the requirements of specific applications of embodiments of the invention. As the RNA-Seq data may describe 15000+ genes, translation into the latent space both acts as a dimensionality reduction to reduce complexity, but further enables the capture of cancer characteristics in the latent space for subsequent processing.

The latent representation of the RNA-Seq data is provided to an RNA-to-Image Diffusion Model which translates the latent representation into a WSI tile image. A Super-Resolution Diffusion Model can then be used to upscale the synthetic WSI tile image to a higher resolution. An advantage of the diffusion model approach as opposed to a generative adversarial network (GAN) approach to generating the synthetic image is that GAN models will tend to collapse on a single class due to the relative homogeneity of tissue. Diffusion models can handle the minute differences in a WSI at the cellular level. In many embodiments, the resulting WSI tiles that are produced maintain the cell distribution of real tiles associated with a given test RNA-Seq data. This architecture is referred to herein as RNA-CDM, a cascaded diffusion model for multi-cancer RNA-to-image synthesis.

In many embodiments, RNA-CDM is used to generate a library of training data for deep learning models that are subsequently used to identify, diagnose and/or prognose cancers. In various embodiments, deep learning models are pre-trained using RNA-CDM generated images to yield a template model which can be subsequently trained again on natural and/or additional synthetic data to produce a specialized model for clinical purposes. In numerous embodiments, RNA-CDM generated images alone or in addition to a number of real data images are used to train a deep learning model for a specialized purpose. In order to construct an RNA-CDM, a number of models need to be trained: the VAE encoder; the RNA-to-Image Diffusion Model; and the Super-Resolution Diffusion Model. Training processes are described below, followed by a discussion of the RNA-CDM architecture.

### Model Training

In many embodiments, training the RNA-CDM models occurs in two phases: 1) training the VAE encoder; and 2) training the diffusion models. VAEs are composed of two separate networks, an encoder and a decoder. The encoder maps the input to a latent space, and the decoder reconstructs the input from the latent space. The driving concept behind the original autoencoder architecture is to learn a smaller representation of the input data by learning the function h_{θ}(x)≈x being θ the parameters of the neural network. In numerous embodiments, the goal is to minimize the reconstruction error between the input and the output of the VAE. The VAE architecture extends this approach to learn a probability distribution of the latent space. The assumption of the VAE is that the distribution of the data x, P(x) is related to the distribution of the latent variable z, P(z). The loss function of the VAE, which is the negative log-likelihood with a regularizer is formalized as:

Lᵢ(θ,ϕ)=− [symbol] _{z˜q}_{_{θ}}_{(z|x}_{ᵢ}₎[log p_{ϕ}(xᵢ|z)]+ [symbol] (q_{θ}(z|xᵢ)∥p(z))

where the first term is the reconstruction loss and the second term is the Kullback-Leibler (KL) divergence between the encoder's distribution q_{θ}(z|xᵢ) and p(z) which is defined as the standard normalized distribution p(z)=N(0,1).

In many embodiments, β-VAE is selected as the VAE architecture, which introduces the parameter β which controls the effect of the KL divergence part of the equation:

Lᵢ(θ,ϕ)=− [symbol] _{z˜q}_{_{θ}}_{(z|x}_{ᵢ}₎[log p_{ϕ}(xᵢ|z)]+β× [symbol] (q_{θ}(z|xᵢ)∥p(z))

If β−1, it is the standard loss of the VAE. If β=0, then there is only a focus on reconstruction loss, approximating the model to a normal autoencoder. In many embodiments, the effect of the KL divergence can be regularized on the training of the model which can result in a smoother and more disentangled latent space. In numerous embodiments, β=0.005, and LeakyReLU is used as the activation function.

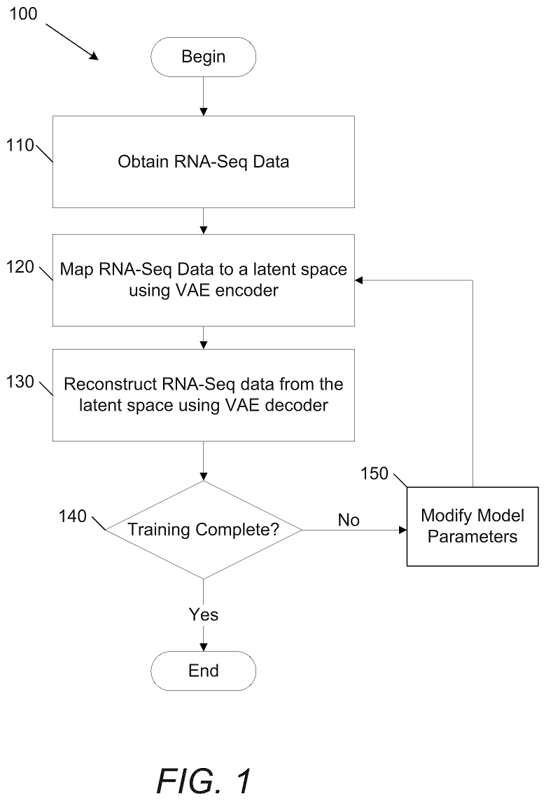

The selected VAE model (e.g. B-VAE) is trained on RNA-Seq data. Turning now to FIG. 1, a process for training the VAE model in accordance with an embodiment of the invention is illustrated. Process 100 includes obtaining (110) RNA-Seq data. The VAE encoder maps (120) the RNA-Seq data to a latent space. The VAE decoder then reconstructs (130) the RNA-Seq data from the latent space. If the training process is complete, then the models are deemed trained. Otherwise, the model parameters are modified (150) and the process continues. In numerous embodiments, the VAE is trained for a predetermined number of epochs with early stopping based on validation set loss. In many embodiments, the VAE is trained between 200 and 300 epochs with a batch size of 128. In a variety of embodiments, an Adam optimizer is used for training with a learning rate equal to 3×10⁻³, along with a warm-up and a cosine learning rate schedule and the mean square error as the loss function. However, as can readily be appreciated, any number of different training parameters can be used to generate a VAE model that performs as noted above to produce a lower-dimension latent space for RNA-Seq data conducive to high accuracy reconstruction.

Once the VAE model is trained, the trained VAE encoder model can be extracted and used for diffusion model training and construction of the RNA-CDM. Diffusion models are a kind of score-based generative model that model the gradient of the log probability density function using score matching. The idea for diffusion models is to learn a series of state transitions to map noise e from a known prior distribution to x₀ from the data distribution. To construct the diffusion models, first an additive noise forward process from x₀ to xₜ is defined as:

xₜ=\sqrt{γ(t)x₀}+\sqrt{1−γ(t)ϵ}

where ϵ˜ [symbol] (0, I), t˜ [symbol] (0, t), and γ(t) is a monotonically decreasing function from 1 to 0. A neural network is learned, {(xₜ, t), to reverse this process by predicting x₀ (or ϵ) from xₜ. In many embodiments, the training of the neural network is based on denoising with an I₂ regression loss:

[symbol]

ₓ_{₀}= [symbol] ∥f(\sqrt{γ(t)x₀}+\sqrt{1−γ(t)ϵ},t)−ϵ∥²

Once the new model is learned, new samples can be generated by reversely going from xₜ→xₜ₋ₙ→ ⋅ ⋅ ⋅ x₀. This can be achieved by applying the denoising function f to the samples to obtain x₀, and then make the transition to xₜ₋ₙ by using the predicted \hat{x}₀.

Cascaded diffusion models have been proposed as a way to improve sample quality. Having high-resolution data x₀ and a low-resolution version z₀, a diffusion model at the low resolution p_{θ}(x₀), and a super-resolution diffusion model p_{θ}(x₀|z₀) can be constructed. The cascading pipeline forms a latent variable model for high resolution data, that can also be extended to conditioning to the class (or the gene expression latent representation in the instant context):

p_{θ}(x₀)=∫p_{θ}(x₀|z₀,c)p_{θ}(z₀,c)dz₀

where c is the gene expression latent representation.

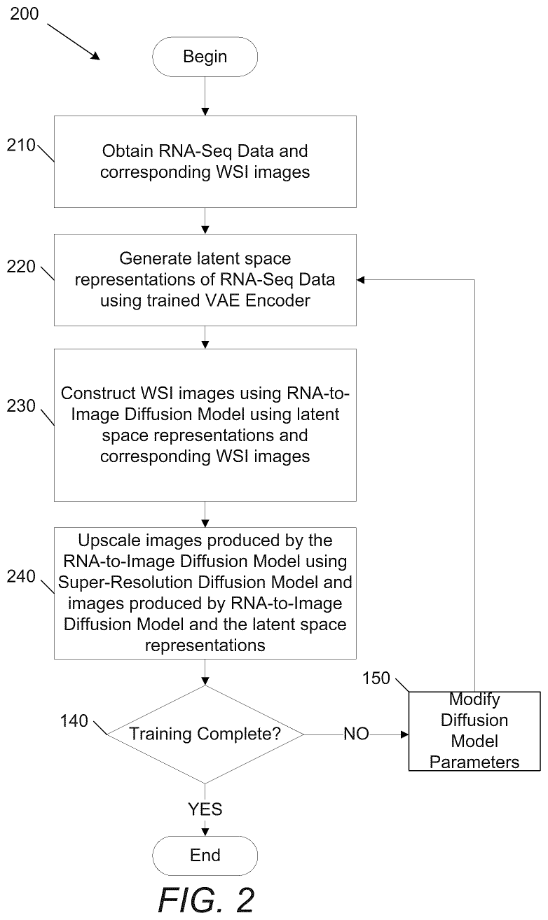

Turning now to FIG. 2, a process for training the RNA-to-Image Diffusion Model and the Super-Resolution Diffusion Model in accordance with an embodiment of the invention is illustrated. Process 200 includes obtaining (210) RNA-Seq data and a set of corresponding WSI images, such that each WSI image is associated with the RNA-Seq data produced by the tissue in the WSI image. The trained VAE encoder is used to generate (220) a latent space representation of the RNA-Seq data. An RNA-to-Image Diffusion Model is used to construct (230) a WSI image by providing the model with a latent space representation of RNA-Seq data and the corresponding WSI. The generated low-resolution images are provided to the Super-Resolution Diffusion Model along with the respective latent space representations of the RNA-Seq data, which in turn produces (240) upscaled WSI images. If the training process is complete (140), then the process ends. If the training process is not complete, then the diffusion model parameters are modified (150) and the process continues.

In many embodiments, the Unet model architecture is used as the diffusion model, using a dimension of 128 for both the low-resolution and the super-resolution diffusion models. In various embodiments, attention and skip connections are used across Unet layers. Adam can be used as the optimizer with a learning rate equal to 1 e 4, and an exponential moving average using during training. In various embodiments, the low-resolution RNA-to-Image Diffusion Model produces a 64×64 image. A Gaussian blur can be applied to the image, and the blurred image is provided to the Super-Resolution Diffusion Model to generate a 256×256 version of the image. The models utilize timesteps=1000 with a linear Gaussian diffusion process, and each model is trained for approximately 50000-55000 steps, with a stopping point decided based on the visual quality of the generated tissue. In various embodiments, the models can be fine-tuned for 10-60 epochs using the AdamW optimizer with a learning rate equal to between 3e² and 3e⁶.

While particular training parameters and model architectures are discussed above, as can be readily appreciated, different diffusion model architectures and VAE encoders can be selected without departing from the scope or spirit of the invention. Similarly, training parameters may be tuned as appropriate to the data being used, accuracy, and/or computing resources available. Following the training of the VAE encoder and the two diffusion models, the RNA-CDM architecture can be constructed. The RNA-CDM architecture is discussed below.

### RNA-CDM

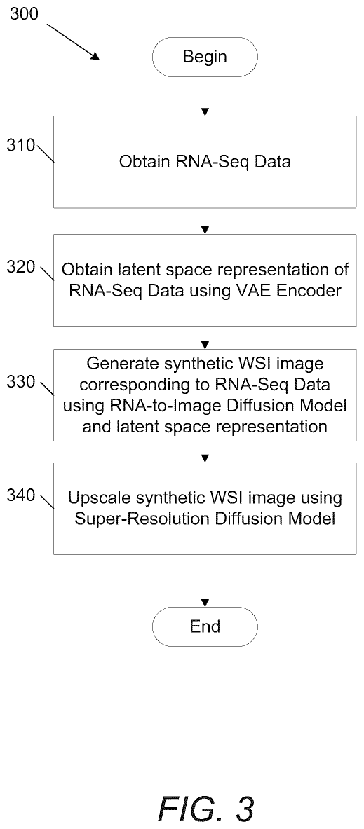

The RNA-CDM architecture includes the three trained models described above. The resulting stack of models is capable of taking in RNA-Seq data and producing high-resolution WSI images that approximate WSI images of real tissue that would produce the input RNA-Seq data. The RNA-CDM architecture is similar to the training architecture for the diffusion models, however it is not necessary to provide sample WSI images. Turning now to FIG. 3, a process for generating synthetic WSI images from RNA-Seq data using RNA-CDM in accordance with an embodiment of the invention is illustrated.

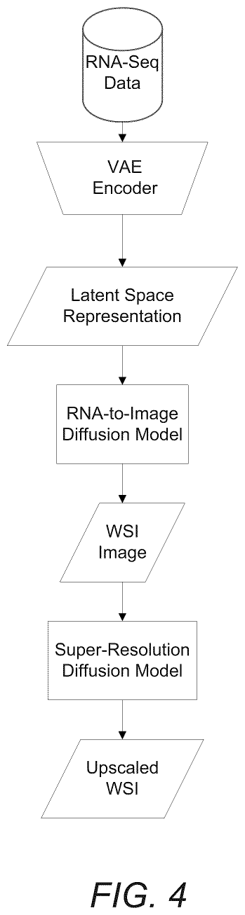

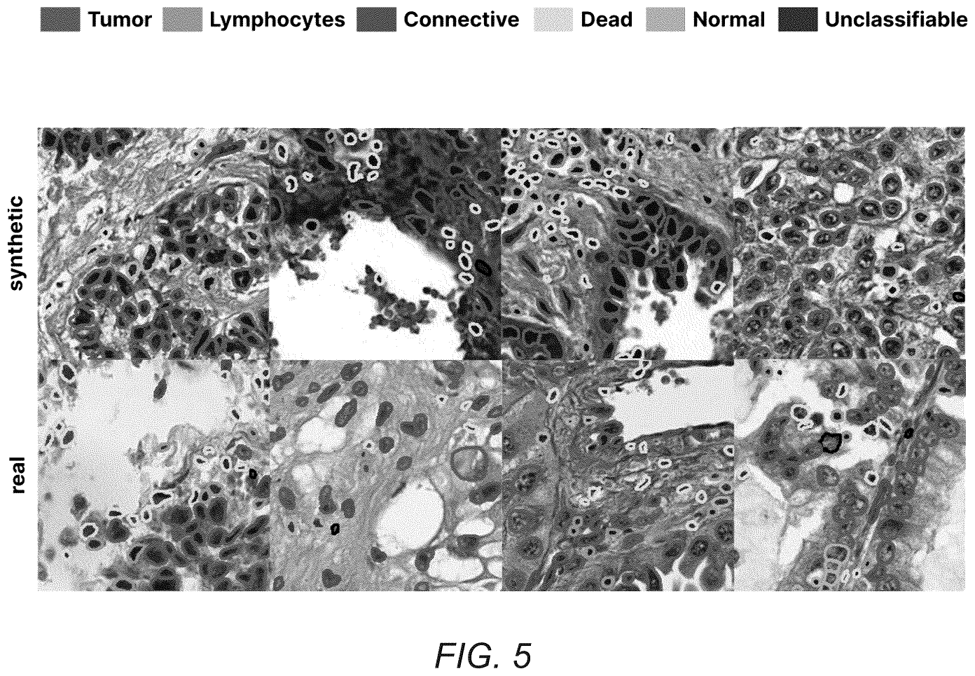

Process 300 includes obtaining (310) RNA-seq data. The trained VAE encoder is used to obtain (320) a latent space representation of the obtained RNA-seq data. The latent space representation is provided to the trained RNA-to-Image Diffusion Model which generates (330) a low-resolution synthetic WSI image. The low-resolution WSI image is then upscaled (340) using the super-resolution diffusion model to produce a high-resolution version of the WSI image. This process is graphically depicted in FIG. 4. A comparison of real WSI images and synthetic WSI images generated using the RNA-Seq data from the corresponding real WSI images are illustrated in FIG. 5, with cell types labeled.

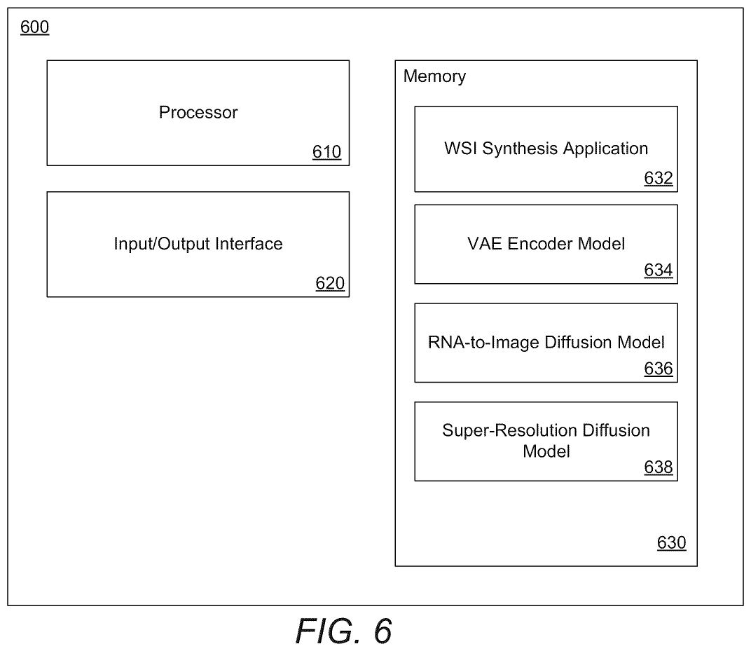

In numerous embodiments, RNA-CDM is implemented using a computing device. When a computing device implements RNA-CDM, it can be referred to as a WSI synthesizer. Turning now to FIG. 6, a WSI synthesizer in accordance with an embodiment of the invention is illustrated. WSI synthesizer 600 includes a processor 610. Processors can be a central processing unit (CPU), a graphics processing unit (GPU), an application-specific integrated circuit (ASIC), field-programable gate array (FPGA), and/or any other logic circuitry as appropriate to the requirements of specific applications of embodiments of the invention. In various embodiments, WSI synthesizers have more than one processor. WSI synthesizer 600 further includes an input/output interface capable of providing and/or receiving data and/or commands to connected devices, and a memory 630. Memory can be volatile memory, non-volatile memory, or any combination thereof.

Memory 630 contains a WSI synthesis application 632 which can configure the processor to execute WSI synthesis processes such as those described herein. The memory 630 further contains the VAE encoder model 634, RNA-to-Image Diffusion Model 636, and Super-Resolution Diffusion Model 638 which make up the RNA-CDM architecture. In numerous embodiments, WSI synthesizers can be used to train the models.

Although specific methods of synthetic WSI image generation and specific WSI synthesizer architectures are discussed above, many different methods and system architectures can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method of generating synthetic histological slide images, comprising:
obtaining a plurality of RNA-Seq records;
obtaining a plurality of histological slide images, where each histological slide image is associated with one of the RNA-Seq records;
translating each record in the plurality of RNA-Seq records into a latent space using an encoder component of a variational auto encoder;
training a first diffusion model to produce a first synthetic histological slide image at a lower resolution using the translated plurality of RNA-Seq records and the associated histological slides;
training a second diffusion model to upscale lower resolution synthetic histological slide images to higher resolution synthetic histological slide images using lower resolution images produced by the first diffusion model and the associated histological slide images;
obtaining a given RNA-Seq record;
translating the given RNA-Seq record into the latent space using the encoder component of the variational autoencoder;
providing the latent representation of the given RNA-Seq record to the trained first diffusion model;
generating a given lower resolution synthetic histological slide image using the trained first diffusion model;
providing the given lower resolution synthetic histological slide image to the trained second diffusion model; and
generating a given higher resolution synthetic histological slide image using the trained second diffusion model.

2. The method of claim 1, wherein the variational autoencoder is a 3-VAE encoder model.

3. The method of claim 1, further comprising training the first and second diffusion models on a second plurality of RNA-Seq records, where each of the RNA-Seq records in the second plurality of RNA-Seq records are associated with one image of a second plurality of histological slide images, and where the second plurality of RNA-Seq records are associated with a specific cancer classification.

4. The method of claim 1, wherein the first and second diffusion models comprise a UNet architecture.

5. The method of claim 1, wherein the lower resolution is 64×64 pixels.

6. The method of claim 1, wherein the higher resolution is 256×256 pixels.

7. The method of claim 1, wherein the given higher resolution synthetic histological slide image is a tile of a larger synthetic histological slide image.

8. The method of claim 7, further comprising generating a plurality of higher resolution synthetic histological slide images; and combining the plurality of higher resolution synthetic histological slide images to form the larger synthetic histological slide image.

9. The method of claim 1, wherein the synthetic histological slide image depicts a plurality of human tissue types.

10. The method of claim 1, further comprising training the encoder component of the variational autoencoder using a decoder component of the variational autoencoder, wherein the encoder component and the decoder component are trained together to minimize reconstruction error at the output of the decoder component.

11. A system for generating synthetic histological slide images, comprising:
a processor; and
a memory, the memory containing a whole-slide image synthesis application that configures the processor to:
obtain an RNA-Seq record;
translate the RNA-Seq record into the latent space using an encoder component of a variational autoencoder;
provide the latent representation of the RNA-Seq record to a first diffusion model;
generate a lower resolution synthetic histological slide image using a trained first diffusion model;
provide the given lower resolution synthetic histological slide image to a second diffusion model; and
generate a given higher resolution synthetic histological slide image using the second diffusion model.

12. The system of claim 11, wherein the encoder component is trained using a decoder component of the variational autoencoder, wherein the encoder component and the decoder component are trained together to minimize reconstruction error at the output of the decoder component.

13. The system of claim 11, wherein the first diffusion model and second diffusion model are trained by:
obtaining a plurality of RNA-Seq records;
obtaining a plurality of histological slide images, where each histological slide image is associated with one of the RNA-Seq records;
translating each record in the plurality of RNA-Seq records into a latent space using an encoder component of a variational auto encoder;
training a first diffusion model to produce a first synthetic histological slide image at a lower resolution using the translated plurality of RNA-Seq records and the associated histological slides; and
training a second diffusion model to upscale lower resolution synthetic histological slide images to higher resolution synthetic histological slide images using lower resolution images produced by the first diffusion model and the associated histological slide images.

14. The system of claim 13, further comprising training the first and second diffusion models on a second plurality of RNA-Seq records, where each of the RNA-Seq records in the second plurality of RNA-Seq records are associated with one image of a second plurality of histological slide images, and where the second plurality of RNA-Seq records are associated with a specific cancer classification.

15. The system of claim 11, wherein the variational autoencoder is a B-VAE encoder model.

16. The system of claim 11, wherein the first and second diffusion models comprise a UNet architecture.

17. The system of claim 11, wherein the lower resolution is 64×64 pixels.

18. The system of claim 11, wherein the higher resolution is 256×256 pixels.

19. The system of claim 11, wherein the given higher resolution synthetic histological slide image is a tile of a larger synthetic histological slide image.

20. The system of claim 19, further comprising generating a plurality of higher resolution synthetic histological slide images; and combining the plurality of higher resolution synthetic histological slide images to form the larger synthetic histological slide image.
